(19) United States
(12) Patent Application Publication
(10) Pub. No.: US 2011/0098573 A1
(43) Pub. Date: Apr. 28, 2011

(54) SYSTEMS AND METHODS FOR COUPLING A TRANSDUCER TO A CONTROL MODULE OF AN INTRAVASCULAR ULTRASOUND IMAGING SYSTEM

(75) Inventors: Peter Thornton, Los Altos, CA (US); John D. Marshall, Los Gatos, CA (US)

(73) Assignee: Boston Scientific SciMed, Inc., Maple Grove, MN (US)

(21) Appl. No.: 12/911,517

(22) Filed: Oct. 25, 2010

Related U.S. Application Data
(60) Provisional application No. 61/255,347, filed on Oct. 27, 2009.

(51) Int. Cl. A61B 8/00

(52) U.S. Cl. 600/467

(57) ABSTRACT

A catheter assembly for an intravascular ultrasound system includes an ultrasound transducer disposed in an image device housing within a lumen of a catheter. The ultrasound transducer transforms applied electrical signals to acoustic signals within a frequency bandwidth centered at an operational frequency and having variable electrical impedances over one or more frequencies within the bandwidth. A distal drive cable is coupled to the imaging device housing. A connector housing couples the distal drive cable to a proximal drive cable. A transducer conductor electrically couples the transducer to a proximal end of the catheter. At least one tuning element is electrically coupled to the transducer conductor. The at least one tuning element matches electrical impedances of the transducer conductor to the ultrasound transducer over at least a subset of frequencies within the frequency bandwidth of the ultrasound transducer.

### CROSS-REFERENCE TO RELATED APPLICATIONS

This application claims the benefit under 35 U.S.C. §119(e) of U.S. Provisional Patent Application Ser. No. 61/255,347 filed on Oct. 27, 2009, which is incorporated herein by reference.

### TECHNICAL FIELD

The present invention is directed to the area of intravascular ultrasound imaging systems and methods of making and using the systems. The present invention is also directed to intravascular ultrasound imaging systems having improved transducer connection systems for coupling transducers to control modules, as well as methods for making and using the intravascular ultrasound imaging systems, transducers, and control modules.

### BACKGROUND

Intravascular ultrasound (“IVUS”) imaging systems have proven diagnostic capabilities for a variety of diseases and disorders. For example, IVUS imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic signals that are transmitted through patient tissue. Reflected pulses of the transmitted acoustic signals are absorbed by the one or more transducers and transformed to electric pulses. The transformed electric pulses are delivered to the image processor and converted to an image displayable on the monitor.

### BRIEF SUMMARY

In one embodiment, a catheter assembly for an intravascular ultrasound system includes a catheter having a length along a longitudinal axis, a distal end, and a proximal end. The catheter defines a lumen extending along at least a portion of the catheter. An imaging device housing is disposed in the lumen of the catheter proximate to the distal end of the catheter. At least one ultrasound transducer is disposed in the imaging device housing. The at least one ultrasound transducer is configured and arranged for transforming applied electrical signals to acoustic signals within a frequency bandwidth centered at an operational frequency and having variable electrical impedances over one or more frequencies within the bandwidth, transmitting the acoustic signals, receiving corresponding echo signals, and transforming the received echo signals to electrical signals. A distal drive cable having a distal end is coupled to the imaging device housing. A proximal drive cable having a proximal end extends to a proximal end of the catheter. A connector housing couples the distal drive cable to the proximal drive cable. At least one transducer conductor is electrically coupled to the at least one transducer and in electrical communication with the proximal end of the catheter. At least one tuning element is electrically coupled to the at least one transducer conductor. The at least one tuning element is configured and arranged to match the electrical impedances of the at least one transducer conductor to the at least one ultrasound transducer over at least a subset of frequencies within the frequency bandwidth of the at least one ultrasound transducer.

In another embodiment, a catheter assembly for an intravascular ultrasound system includes a catheter having a length along a longitudinal axis, a distal end, and a proximal end. The catheter defines a lumen extending along at least a portion of the catheter. An imaging device housing is disposed in the lumen of the catheter proximate to the distal end of the catheter. At least one ultrasound transducer is disposed in the imaging device housing. The at least one ultrasound transducer is configured and arranged for transforming applied electrical signals to acoustic signals within a frequency bandwidth centered at an operational frequency and having variable electrical impedances over one or more frequencies within the bandwidth, transmitting the acoustic signals, receiving corresponding echo signals, and transforming the received echo signals to electrical signals. A distal drive cable having a distal end is coupled to the imaging device housing. The distal drive cable has a first torsional stiffness. A proximal drive cable having a proximal end extends to a proximal end of the catheter. The proximal drive cable has a second torsional stiffness. The second torsional stiffness is substantially greater than the first torsional stiffness. A connector housing couples the distal drive cable to the proximal drive cable. At least one transducer conductor is electrically coupled to the at least one transducer and in electrical communication with the proximal end of the catheter.

In yet another embodiment, a method for imaging a patient using an intravascular ultrasound imaging system includes inserting a catheter assembly into patient vasculature. The catheter assembly includes a catheter defining a lumen extending along at least a portion of the catheter. At least one ultrasound transducer is disposed in an imaging device housing within the lumen. A distal drive cable having a distal end is coupled to the imaging device housing. A proximal drive cable having a proximal end is coupled to a control module. A connector housing couples the distal drive cable to the proximal drive cable. The at least one ultrasound transducer is positioned in proximity to a region to be imaged. At least one electrical signal is transmitted from the control module to the at least one transducer via at least one transducer conductor. Acoustic signals are transmitted from the at least one transducer to patient tissue. The acoustic signals have variable electrical impedances over a frequency bandwidth centered at an operational frequency. At least one echo signal is received from a tissue-boundary between adjacent imaged patient tissue by the imaging core. At least one transformed echo signal is transmitted from the at least one transducer to the control module for processing via the at least one transducer conductor. The at least one transformed echo signal propagates through at least one tuning element. The at least one tuning element is configured and arranged to match the electrical impedances of the at least one transducer conductor to the at least one ultrasound transducer over at least a subset of frequencies within the frequency bandwidth of the at least one ultrasound transducer.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4 is a schematic perspective view of one embodiment of the imaging core of FIG. 3, the imaging core including a connector housing coupling a distal drive cable to a proximal drive cable, according to the invention;

FIG. 5 is a schematic perspective view of one embodiment of tuning elements disposed in the connector housing of FIG. 5, according to the invention;

FIG. 6A is a schematic diagram of one embodiment of a portion of an imaging circuit for an intravascular ultrasound imaging system, the imaging circuit including tuning elements disposed between a transducer and a control module, according to the invention; and

FIG. 6B is a schematic diagram of another embodiment of a portion of an imaging circuit for an intravascular ultrasound imaging system, the imaging circuit including tuning elements disposed between a transducer and a control module, according to the invention.

### DETAILED DESCRIPTION

The present invention is directed to the area of intravascular ultrasound imaging systems and methods of making and using the systems. The present invention is also directed to intravascular ultrasound imaging systems having improved transducer connection systems for coupling transducers to control modules, as well as methods for making and using the intravascular ultrasound imaging systems, transducers, and control modules.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; and 6,945,938; as well as U.S. Patent Application Publication Nos. 20060253028; 20070016054; 20070038111; 20060173350; and 20060100522, all of which are incorporated by reference.

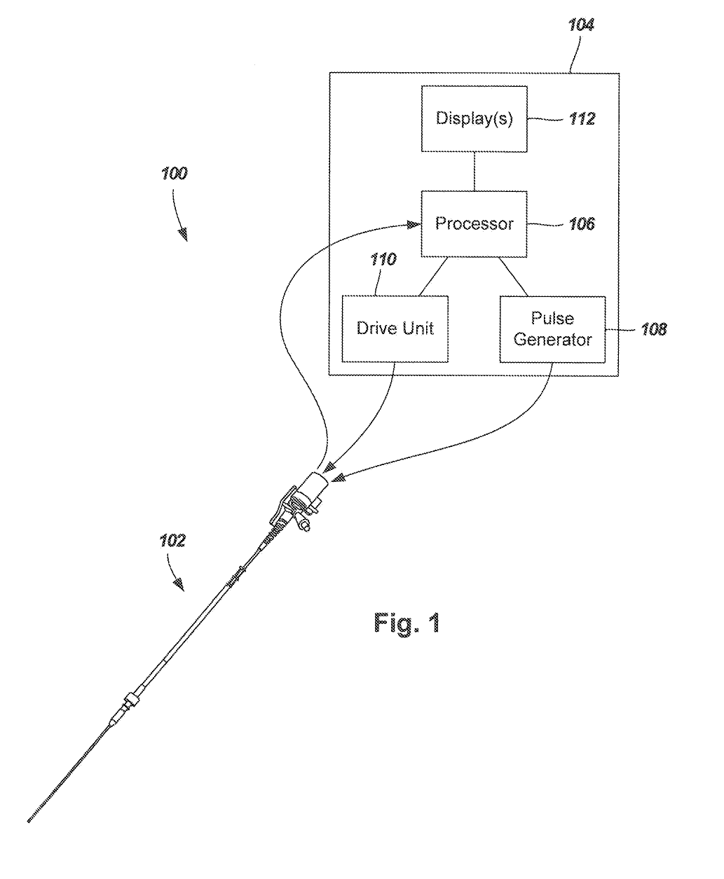

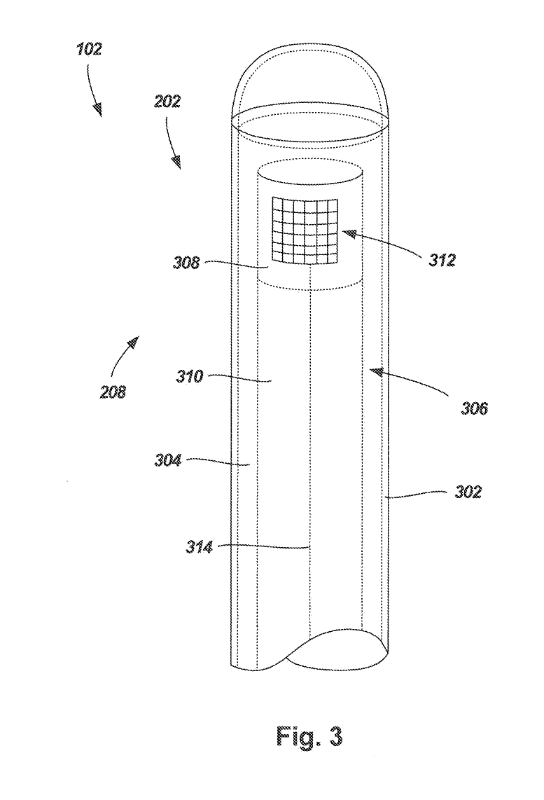

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric signals that may be input to one or more transducers (312 in FIG. 3) disposed in the catheter 102. In at least some embodiments, mechanical energy from the drive unit 110 may be used to drive an imaging core (306 in FIG. 3) disposed in the catheter 102. In at least some embodiments, electric signals transmitted from the one or more transducers (312 in FIG. 3) may be input to the processor 106 for processing. In at least some embodiments, the processed electric signals from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical signals transmitted from the pulse generator 108, the rotation rate of the imaging core (306 in FIG. 3) by the drive unit 110, the velocity or length of the pullback of the imaging core (306 in FIG. 3) by the drive unit 110, or one or more properties of one or more images formed on the one or more displays 112.

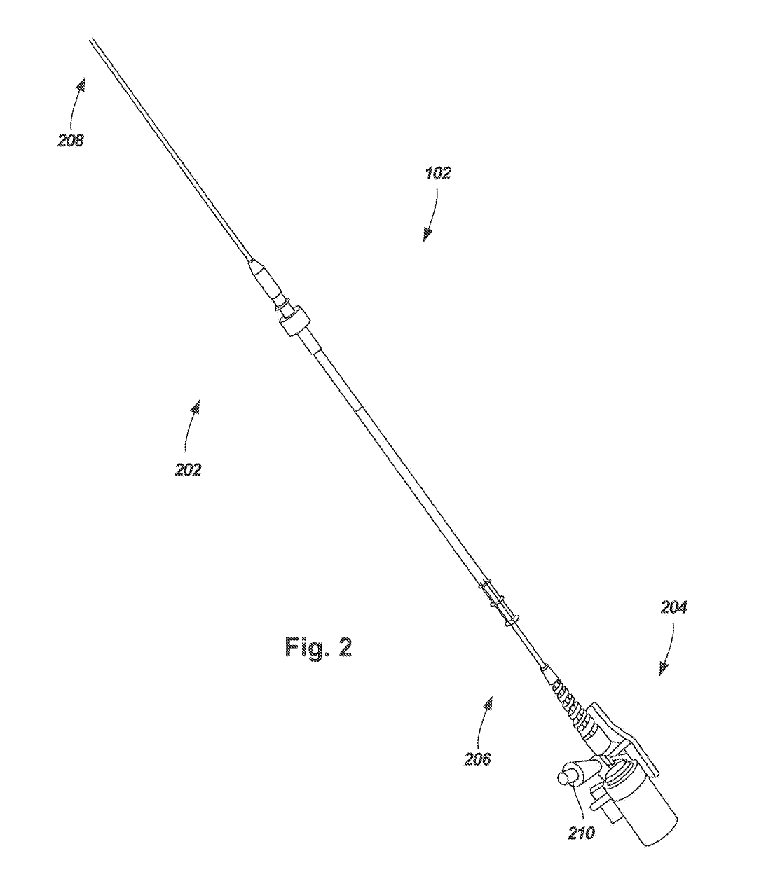

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device housing 308 coupled to a distal end of a transducer connection system, such as a drive cable 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device housing 308 and employed to transmit and receive acoustic signals. In a preferred embodiment (as shown in FIG. 3), an array of transducers 312 are mounted to the imaging device housing 308. In other embodiments, a single transducer may be employed. In yet other embodiments, multiple transducers in an irregular-array may be employed. Any number of transducers 312 can be used. For example, there can be one, two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

The one or more transducers 312 may be formed from one or more known materials capable of transforming applied electrical signals to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like.

The pressure distortions on the surface of the one or more transducers 312 form acoustic signals of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic signals of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic signals.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

In at least some embodiments, the imaging core 306 may be rotated about a longitudinal axis of the catheter 102. As the imaging core 306 rotates, the one or more transducers 312 emit acoustic signals in different radial directions. When an emitted acoustic signal with sufficient energy encounters one or more medium boundaries, such as one or more tissue boundaries, a portion of the emitted acoustic signal is reflected back to the emitting transducer as an echo signal. Each echo signal that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic signals transmitted and the echo signals received. In at least some embodiments, the rotation of the imaging core 306 is driven by the drive unit 110 disposed in the control module (104 in FIG. 1) via the transducer connection system 310.

As the one or more transducers 312 rotate about the longitudinal axis of the catheter 102 emitting acoustic signals, a plurality of images are formed that collectively form a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays 112.

In at least some embodiments, the imaging core 306 may also move axially along the blood vessel within which the catheter 102 is inserted so that a plurality of cross-sectional images may be formed along an axial length of the blood vessel. In at least some embodiments, during an imaging procedure the one or more transducers 312 may be retracted (i.e., pulled back) along the longitudinal length of the catheter 102. In at least some embodiments, the catheter 102 includes at least one telescoping section that can be retracted during pullback of the one or more transducers 312. In at least some embodiments, the drive unit 110 drives the pullback of the imaging core 306 within the catheter 102. In at least some embodiments, the drive unit 110 pullback distance of the imaging core is at least 5 cm. In at least some embodiments, the drive unit 110 pullback distance of the imaging core is at least 10 cm. In at least some embodiments, the drive unit 110 pullback distance of the imaging core is at least 15 cm. In at least some embodiments, the drive unit 110 pullback distance of the imaging core is at least 20 cm. In at least some embodiments, the drive unit 110 pullback distance of the imaging core is at least 25 cm.

The quality of an image produced at different depths from the one or more transducers 312 may be affected by one or more factors including, for example, bandwidth, transducer focus, beam pattern, as well as the frequency of the acoustic signal. The frequency of the acoustic signal output from the one or more transducers 312 may also affect the penetration depth of the acoustic signal output from the one or more transducers 312. In general, as the frequency of an acoustic signal is lowered, the depth of the penetration of the acoustic signal within patient tissue increases. In at least some embodiments, the IVUS imaging system 100 transmits acoustic signals centered at an operational frequency that is within a frequency range of 5 MHz to 60 MHz.

In at least some embodiments, the one or more transducers 312 may be mounted to the distal end 208 of the imaging core 306. The imaging core 306 may be inserted in the lumen of the catheter 102. In at least some embodiments, the catheter 102 (and imaging core 306) may be inserted percutaneously into a patient via an accessible blood vessel, such as the femoral artery, at a site remote from a target imaging location. The catheter 102 may then be advanced through patient vasculature to the target imaging location, such as a portion of a selected blood vessel.

As discussed above, the transducer connection system 310 couples the imaging device housing 308 to the control module (104 in FIG. 1). In at least some embodiments, the one or more transducer conductors 314 extend along the transducer connection system 310. In at least some embodiments, one or more transducer conductors 314 electrically couple the one or more transducers 312 to the control module 104 (104 in FIG. 1).

In designing a transducer connection system that utilizes a drive cable, it is useful to consider the torsional stiffness of the drive cable. The drive cable is formed to be torsionally stiff (“stiff”) enough to carry a torque sufficient to rotate the one or more transducers at the distal end of the imaging core, yet flexible enough to maneuver the one or more transducers through potentially tortuous patient vasculature to target imaging locations. It is undesirable for the drive cable to experience substantial “wind up” which occurs as a result of twisting along a length of the drive cable.

Moreover, it is desirable to have sufficient torque to maintain uniform rotation of the imaging core 306 during operation. For example, when the imaging core 306 is pulled back during an imaging procedure, it is desirable for the imaging core 306 to be able to maneuver through tortuous or narrow regions which may press against one or more portions of the imaging core 306 within the catheter 102 without causing a non-uniform rotation (e.g., a wobble, a vibration, a stall, or the like) of the one or more transducers 312 during operation. Non-uniform rotation may lead to the distortion of a subsequently-generated IVUS image (e.g., the subsequently-generated IVUS image may be blurred).

In at least some embodiments, a transducer connection system utilizes a distal drive cable and a proximal drive cable axially coupled to one another and extendable along at least a portion of the lumen of the catheter. In at least some embodiments, the proximal drive cable is coupled to the distal drive cable via a connector housing. In at least some embodiments, one or more tuning elements are in electrical communication with the one or more transducer conductors to improve signal propagation efficiency or reduce noise or both. In at least some embodiments, the one or more tuning elements can be disposed in the imaging device housing 308. In preferred embodiments, the one or more tuning elements are disposed in the connector housing.

FIG. 4 is a schematic perspective view of one embodiment of the imaging core 306. The imaging core 306 includes the imaging device housing 308 and the transducer connection system 310. The transducer connection system 310 includes a distal drive cable 402, a proximal drive cable 404, and a connector housing 406. The distal drive cable 402 couples the imaging device housing 308 to the connector housing 406. The proximal drive cable 404 extends proximally from the connector housing 406. In at least some embodiments, the proximal drive cable 404 couples the connector housing 406 to the control module (104 in FIG. 1). In FIG. 4, the connector housing 406 couples a distal end of the proximal drive cable 404 to a proximal end of the distal drive cable 402. In some embodiments, the distal drive cable 402 and the proximal drive cable 404 have the same torsional stiffness. In other embodiments, the distal drive cable 402 and the proximal drive cable 404 have different torsional stiffnesses.

Some conventional imaging cores utilize a drive cable that includes a single counterwound coil along a length of the drive cable. With a single counterwound coil, for a given imaging procedure, in order to design a drive cable that is stiff enough to carry a torque sufficient to uniformly rotate the one or more transducers, one or more portions of the drive cable may not be flexible enough to maneuver the one or more transducers through the patient to a target imaging location.

It may be an advantage to design a transducer connection system having a plurality of different stiffnesses. For example, a proximal end of the transducer connection system can be designed to be stiff enough to carry a torque sufficient to uniformly rotate one or more transducers, while a distal end of the transducer connection system can be designed to be flexible enough to maneuver the one or more transducers through patient vasculature to a desired imaging location.

In at least some embodiments, the proximal drive cable 404 is at least 5% more torsionally stiff than the distal drive cable 402. In at least some embodiments, the proximal drive cable 404 is at least 10% more torsionally stiff than the distal drive cable 402. In at least some embodiments, the proximal drive cable 404 is at least 15% more torsionally stiff than the distal drive cable 402. In at least some embodiments, the proximal drive cable 404 is at least 20% more torsionally stiff than the distal drive cable 402. In at least some embodiments, the proximal drive cable 404 is at least 25% more torsionally stiff than the distal drive cable 402. In at least some embodiments, the proximal drive cable 404 is at least 30% more torsionally stiff than the distal drive cable 402. In at least some embodiments, the proximal drive cable 404 is at least 40% more torsionally stiff than the distal drive cable 402. In at least some embodiments, the proximal drive cable 404 is at least 50% more torsionally stiff than the distal drive cable 402.

In at least some embodiments, the distal drive cable 402 and the proximal drive cable 404 have equal lengths. In at least some embodiments, the proximal drive cable 404 is longer than the distal drive cable 402. In at least some embodiments, the proximal drive cable 404 is at least 10% longer than the distal drive cable 402. In at least some embodiments, the proximal drive cable 404 is at least 20% longer than the distal drive cable 402. In at least some embodiments, the proximal drive cable 404 is at least 30% longer than the distal drive cable 402. In at least some embodiments, the proximal drive cable 404 is at least 40% longer than the distal drive cable 402. In at least some embodiments, the proximal drive cable 404 is at least 50% longer than the distal drive cable 402. In at least some embodiments, the proximal drive cable 404 is at least 100% longer than the distal drive cable 402.

In at least some embodiments, at least one of the distal drive cable 402 or the proximal drive cable 404 are multifilar. In at least some embodiments, the distal drive cable 402 has six filaments. In at least some embodiments, the distal drive cable 402 has seven filaments. In at least some embodiments, the distal drive cable 402 has eight filaments. In at least some embodiments, the distal drive cable 402 has nine filaments. In at least some embodiments, the distal drive cable 402 has ten filaments.

In at least some embodiments, the proximal drive cable 404 has six filaments. In at least some embodiments, the proximal drive cable 404 has seven filaments. In at least some embodiments, the proximal drive cable 404 has eight filaments. In at least some embodiments, the proximal drive cable 404 has nine filaments. In at least some embodiments, the proximal drive cable 404 has ten filaments. In at least some embodiments, the proximal drive cable 404 has eleven filaments. In at least some embodiments, the proximal drive cable 404 has twelve filaments. In at least some embodiments, the proximal drive cable 404 has thirteen filaments. In at least some embodiments, the proximal drive cable 404 has fourteen filaments. In at least some embodiments, the proximal drive cable 404 has fifteen filaments. In at least some embodiments, the proximal drive cable 404 has sixteen filaments. It will be understood that, in at least some embodiments, the proximal drive cable 404 includes more than sixteen filaments.

In some embodiments, the distal drive cable 402 and the proximal drive cable 404 are the both multifilar. In at least some embodiments, the proximal drive cable 404 is formed from a multifilar material that has a the same number of filaments as the distal drive cable 402. In at least some embodiments, the proximal drive cable 404 is formed from a multifilar material that includes more filaments than the distal drive cable 402.

In some embodiments, the distal drive cable 402 and the proximal drive cable 404 have equal diameters. In other embodiments, the proximal drive cable 404 has a larger diameter than the distal drive cable 402. In other embodiments, the proximal drive cable 404 has a diameter that is no more than 5% larger than the distal drive cable 402. In other embodiments, the proximal drive cable 404 has a diameter that is no more than 10% larger than the distal drive cable 402. In other embodiments, the proximal drive cable 404 has a diameter that is no more than 15% larger than the distal drive cable 402. In other embodiments, the proximal drive cable 404 has a diameter that is no more than 20% larger than the distal drive cable 402.

In at least some embodiments, the proximal drive cable 404 has a transverse cross-sectional profile that is round. In at least some embodiments, the proximal drive cable 404 has a transverse cross-sectional profile that is rectangular. In at least some embodiments, at least one of the distal drive cable 402 or the proximal drive cable 404 is formed from a solid conductive tube formed from a kink-resistant conductive material (e.g., nitinol, or the like).

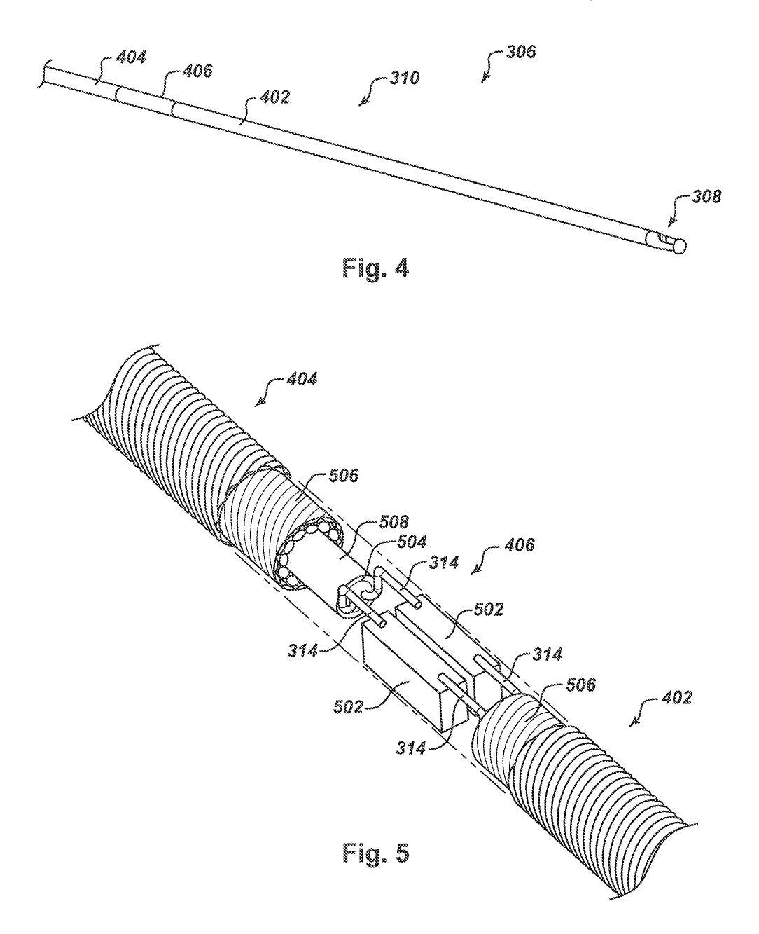

In at least some embodiments, the connector housing 406 has a transverse cross-sectional profile that is the same shape as at least one of the distal drive cable 402 or the proximal drive cable 404. In at least some embodiments, the connector housing 406 has a transverse cross-sectional profile that is round. In at least some embodiments, the connector housing 406 has a diameter that is large enough to house one or more tuning elements (502 in FIG. 5).

In at least some embodiments, the connector housing 406 has a diameter that is equal to the diameter of the proximal drive cable 404. In at least some embodiments, the connector housing 406 has a diameter that is larger than the diameter of the proximal drive cable 404. In at least some embodiments, the connector housing 406 has a diameter that is no more than 5% larger than the diameter of the proximal drive cable 404. In at least some embodiments, the connector housing 406 has a diameter that is no more than 10% larger than the diameter of the proximal drive cable 404. In at least some embodiments, the connector housing 406 has a diameter that is no more than 15% larger than the diameter of the proximal drive cable 404. In at least some embodiments, the connector housing 406 has a diameter that is no more than 20% larger than the diameter of the proximal drive cable 404.

As discussed above, the one or more transducer conductors 314 extend along the transducer connection system 310. In preferred embodiments, the one or more transducer conductors 314 extend within the connector housing 406. In at least some embodiments, the one or more transducer conductors 314 extend within at least a portion of the distal drive cable 402. In at least some embodiments, the one or more transducer conductors 314 extend within at least a portion of the proximal drive cable 404. In at least some embodiments, at least one of the distal drive cable 402 or the proximal drive cable 404 define a lumen through which the one or more transducer conductors 314 extend.

In at least some embodiments, one or more tuning elements are in electrical communication with the one or more transducer conductors 314. The one or more tuning elements are configured and arranged to match, or nearly match, the electrical impedance of the one or more transducer conductors 314 to the one or more transducers 312 over at least a subset of the operational frequency bandwidth of the one or more transducers 312. In at least some embodiments, matching, or nearly matching, the electrical impedance of the one or more transducer conductors 314 to the one or more transducers 312 over at least a subset of the frequency bandwidth of the one or more transducers 312 may improve the efficiency of signal propagation along the propagating along the one or more transducer conductors 314. Electrical noise may be due to a capacitance between the catheter and the body of a patient. Ultrasound images formed by an IVUS imaging system may be degraded by electrical noise. Some conventional systems decrease electrical noise is by increasing the thickness of an insulating one or more transducer conductors 314.

It may be an advantage to design a transducer connection system that utilizes one or more tuning elements to tune the one or more transducer conductors to match, or nearly match, the electrical impedance of the one or more transducer conductors 314 to the one or more transducers 312 over at least a subset of the operational frequency bandwidth of the one or more transducers 312. Tuning the one or more transducer conductors 314 may increase signal propagation efficiency along the one or more transducer conductors 314.

In at least some embodiments, matching, or nearly matching, the electrical impedance of the one or more transducer conductors 314 to the one or more transducers 312 over at least a subset of the operational frequency bandwidth of the one or more transducers 312 may reduce the amount of noise introduced to signals dielectric cover of the one or more transducer conductors 314 or by increasing the amount of space between the one or more transducer conductors 314 and the patient. It is desirable, however, to use a small diameter catheter to increase the number of blood vessels that the one or more coupled transducers 312 may be able to image.

FIG. 5 is a schematic perspective view of one embodiment of one or more tuning elements 502 disposed in the connector housing 406 between the distal drive cable 402 and the proximal drive cable 404. In FIG. 5, an outer covering of the connector housing 406 is shown as being transparent, for clarity of illustration.

The distal drive cable 402 and the proximal drive cable 404 may be structured in many different ways. For example, in at least some embodiments, the proximal drive cable 404 includes a shield 508 and an inner insulator 504. In at least some embodiments, the one or more transducer conductors 314 extend within the inner insulator 504. In at least some embodiments, the inner insulator 504 electrically insulates the one or more transducer conductors 314 from the shield 508. In at least some embodiments, an outer jacket electrically insulates the shield 508 from the proximal drive cable 404 and its surrounding environment. In at least some embodiments, the distal drive cable 402 is arranged in a manner similar to the proximal drive cable 404.

In some embodiments, the one or more tuning elements 502 include an inductor. In at least some embodiments, the one or more tuning elements 502 include a plurality of inductors positioned in series. An example of a suitable inductor for use (alone or in series) in a transducer connection system is an HK-0603 series component from Taiyo Yuden, Tokyo, Japan.

In at least some embodiments, the one or more tuning elements 502 are disposed directly between the one or more transducers 312 and the one or more transducer conductors 314. Disposing the one or more tuning elements 502 in (or adjacent to) the imaging device housing (308 in FIG. 3), however, may increase the size of the imaging device housing (308 in FIG. 3).

It may be an advantage to dispose the one or more tuning elements along the transducer connection system instead of in the imaging device housing 308 because disposing the one or more tuning elements in the imaging device housing 308 may reduce the maneuverability of the imaging core 306 within patient vasculature, thereby potentially reducing reachable target imaging regions within patient vasculature.

It has been found that disposing the one or more tuning elements 502 in proximity to the imaging device housing (308 in FIG. 3) without disposing the one or more tuning elements 502 in (or adjacent to) the imaging device housing (308 in FIG. 3) may obtain much of the benefit of tuning the one or more transducer conductors 314 to match, or nearly match, the electrical impedance of the one or more transducer conductors 314 to the one or more transducers 312 over at least a subset of the operational frequency bandwidth of the one or more transducers 312 without obtaining the adverse effects of reduced maneuverability of the distal end of the catheter within patient vasculature.

In at least some embodiments, the one or more tuning elements 502 are disposed a distance from the one or more transducers 312 that is no greater than one-tenth of a wavelength of at least one frequency of the operational frequency bandwidth of the one or more transducers 312. For example, when a frequency within the operational frequency bandwidth of the one or more transducers 312 is 30 MHz, the wavelength of the transmitted acoustic signals at that frequency are approximately 33 feet (approximately ten meters). Thus, the exemplary frequency of 30 MHz, the one or more tuning elements 502 are disposed a distance from the one or more transducers that is no greater than one-tenth of approximately 33 feet (approximately 10 meters), or approximately 3.3 feet (approximately one meter). In at least some embodiments, when the one or more tuning elements 502 are disposed in the connector housing 406, the length of the distal drive cable 402 is also no greater than one-tenth of wavelengths of a frequency within the operational frequency bandwidth of the one or more transducers 312. It will be understood that the one or more transducers 312 may have many different operational frequency bandwidths that may or may not include 30 MHz.

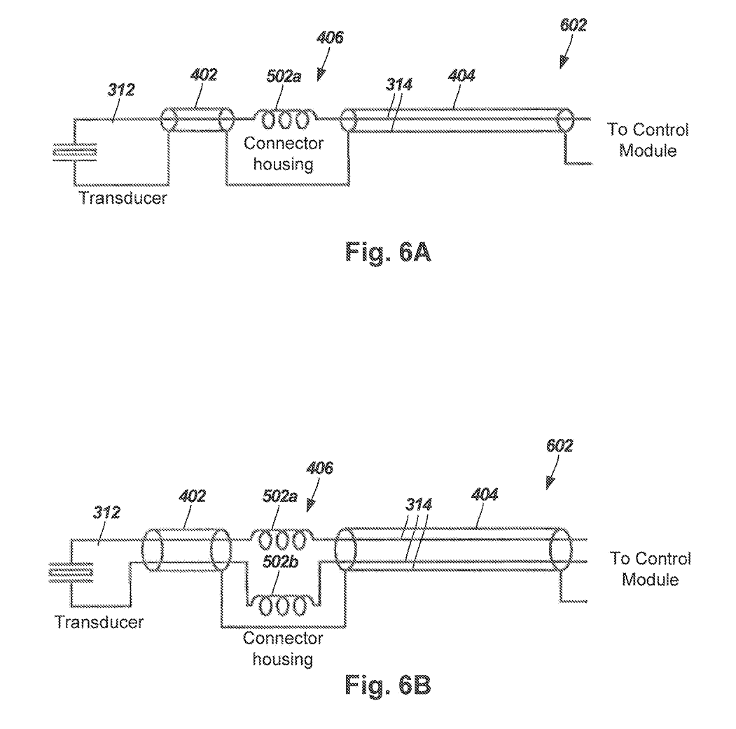

FIGS. 6A-6B are schematic diagrams of exemplary embodiments of a portion of an imaging circuit 602 for the intravascular ultrasound imaging system (100 in FIG. 1). The imaging circuit 602 electrically couples the one or more transducers 312 to the control module 104 via the one or more transducer conductors 314. The imaging circuit 602 includes the one or more tuning elements 502. In FIGS. 6A and 6B, the one or more transducer conductors 314 extend within the distal drive cable 402, the proximal drive cable 404, and the connector housing 406.

In FIGS. 6A and 6B, the one or more tuning elements 502, are disposed in the connector housing 406. In FIG. 6A, the imaging circuit 602 includes a first tuning element 502a. In at least some embodiments, a single-ended transducer connection system, for example a transducer connection system using a coaxial cable as the one or more transducer conductors 314, utilizes the first tuning element 502a. In FIG. 6B, the imaging circuit 602 includes the first tuning element 502a and a second tuning element 502b. In at least some embodiments, a balanced-line transducer connection system, for example a transducer connection system using a twisted pair of conductors as the one or more transducer conductors 314, utilizes a plurality of tuning elements 502a and 502b.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A catheter assembly for an intravascular ultrasound system, the catheter assembly comprising:
a catheter having a length along a longitudinal axis, a distal end, and a proximal end, the catheter defining a lumen extending along at least a portion of the catheter;
an imaging device housing disposed in the lumen of the catheter proximate to the distal end of the catheter;
at least one ultrasound transducer disposed in the imaging device housing, the at least one ultrasound transducer configured and arranged for transforming applied electrical signals to acoustic signals within a frequency bandwidth centered at an operational frequency and having variable electrical impedances over one or more frequencies within the bandwidth, transmitting the acoustic signals, receiving corresponding echo signals, and transforming the received echo signals to electrical signals;
a distal drive cable having a distal end coupled to the imaging device housing;
a proximal drive cable having a proximal end extending to a proximal end of the catheter;
a connector housing coupling the distal drive cable to the proximal drive cable;
at least one transducer conductor electrically coupled to the at least one transducer and in electrical communication with the proximal end of the catheter; and
at least one tuning element electrically coupled to the at least one transducer conductor, the at least one tuning element configured and arranged to match the electrical impedances of the at least one transducer conductor to the at least one ultrasound transducer over at least a subset of frequencies within the frequency bandwidth of the at least one ultrasound transducer.

2. The catheter assembly of claim 1, wherein the at least one tuning element is disposed in the connector housing.

3. The catheter assembly of claim 1, wherein the at least one tuning element comprises an inductor.

4. The catheter assembly of claim 1, wherein the at least one tuning element comprises a plurality of inductors in series.

5. The catheter assembly of claim 1, wherein the at least one tuning element is disposed a distance from the at least one ultrasound transducer that is no greater than one-tenth of a wavelength of a frequency within the operational frequency bandwidth of the at least one ultrasound transducer.

6. An intravascular ultrasound imaging system comprising:
the catheter assembly of claim 1; and
a control module coupled to the imaging core, the control module comprising
a pulse generator configured and arranged for providing electric signals to the at least one transducer, the pulse generator electrically coupled to the at least one transducer via the at least one transducer conductor, and
a processor configured and arranged for processing received electrical signals from the at least one transducer to form at least one image, the processor electrically coupled to the at least one transducer via the at least one transducer conductor.

7. A catheter assembly for an intravascular ultrasound system, the catheter assembly comprising:
a catheter having a length along a longitudinal axis, a distal end, and a proximal end, the catheter defines a lumen extending along at least a portion of the catheter;
an imaging device housing disposed in the lumen of the catheter proximate to the distal end of the catheter;
at least one ultrasound transducer disposed in the imaging device housing, the at least one ultrasound transducer configured and arranged for transforming applied electrical signals to acoustic signals within a frequency bandwidth centered at an operational frequency and having variable electrical impedances over one or more frequencies within the bandwidth, transmitting the acoustic signals, receiving corresponding echo signals, and transforming the received echo signals to electrical signals;
a distal drive cable having a distal end coupled to the imaging device housing, the distal drive cable having a first torsional stiffness;
a proximal drive cable having a proximal end extending to a proximal end of the catheter, the proximal drive cable having a second torsional stiffness, wherein the second torsional stiffness is substantially greater than the first torsional stiffness;
a connector housing coupling the distal drive cable to the proximal drive cable; and
at least one transducer conductor electrically coupled to the at least one transducer and in electrical communication with the proximal end of the catheter.

8. The catheter assembly of claim 7, further comprising at least one tuning element electrically coupled to the at least one transducer conductor, the at least one tuning element configured and arranged to match the electrical impedances of the at least one transducer conductor to the at least one ultrasound transducer over at least a subset of frequencies within the frequency bandwidth of the at least one transducer.

9. The catheter assembly of claim 8, wherein the at least one tuning element is disposed in the connector housing.

10. The catheter assembly of claim 7, wherein the connector housing has a diameter that is no greater than the proximal drive cable.

11. The catheter assembly of claim 7, wherein the connector housing has a diameter that is no more than 10% greater than a diameter of the proximal drive cable.

12. The catheter assembly of claim 7, wherein the distal drive cable and the proximal drive cable have equal diameters.

13. The catheter assembly of claim 7, wherein the proximal drive cable is longer in length than the distal drive cable.

14. The catheter assembly of claim 7, wherein at least one of the distal drive cable or the proximal drive cable is formed from nitinol.

15. The catheter assembly of claim 7, wherein at least one of the distal drive cable or the proximal drive cable is multifilar.

16. The catheter assembly of claim 15, wherein the proximal drive cable and the distal drive cable are both multifilar and the proximal drive cable has a larger number of filaments than the distal drive cable.

17. An intravascular ultrasound imaging system comprising:
the catheter assembly of claim 7; and
a control module coupled to the imaging core, the control module comprising
a pulse generator configured and arranged for providing electric signals to the at least one transducer, the pulse generator electrically coupled to the at least one transducer via the at least one transducer conductor, and
a processor configured and arranged for processing received electrical signals from the at least one transducer to form at least one image, the processor electrically coupled to the at least one transducer via the at least one transducer conductor.

18. A method for imaging a patient using an intravascular ultrasound imaging system, the method comprising:
inserting a catheter assembly into patient vasculature, the catheter assembly comprising a catheter defining a lumen extending along at least a portion of the catheter, at least one ultrasound transducer disposed in an imaging device housing within the lumen, a distal drive cable having a distal end coupled to the imaging device housing, a proximal drive cable having a proximal end coupled to a control module, and a connector housing coupling the distal drive cable to the proximal drive cable;
positioning the at least one ultrasound transducer in proximity to a region to be imaged;
transmitting at least one electrical signal from the control module to the at least one transducer via at least one transducer conductor;
transmitting acoustic signals from the at least one transducer to patient tissue, the acoustic signals having variable electrical impedances over a frequency bandwidth centered at an operational frequency;
receiving at least one echo signal from a tissue-boundary between adjacent imaged patient tissue by the imaging core; and
transmitting at least one transformed echo signal from the at least one transducer to the control module for processing via the at least one transducer conductor, wherein the at least one transformed echo signal propagates through at least one tuning element, the at least one tuning element configured and arranged to match the electrical impedances of the at least one transducer conductor to the at least one ultrasound transducer over at least a subset of frequencies within the frequency bandwidth of the at least one ultrasound transducer.

19. The method of claim 18, wherein inserting the catheter assembly into patient vasculature comprises inserting the catheter assembly into patient vasculature, wherein the at least one tuning element is disposed in the connector housing.

20. The method of claim 18, wherein inserting the catheter assembly into patient vasculature comprises inserting the catheter assembly into patient vasculature, wherein the proximal drive cable of the catheter has a torsional stiffness that is greater than a torsional stiffness of the distal drive cable.
